(19) United States
(12) Patent Application Publication
(10) Pub. No.: US 2026/0315817 A1
(43) Pub. Date: Oct. 15, 2026

(54) TREATMENT OF ATRIAL FIBRILLATION BY ACTIVATION OF ADENOSINE - A2aAR RECEPTOR PATHWAY WITH PULSED ELECTROMAGNETIC FIELD STIMULATION

(71) Applicant: Victor I. CHORNENKY, Minnetonka, MN (US)

(72) Inventor: Victor I. CHORNENKY, Minnetonka, MN (US)

(21) Appl. No.: 19/641,453

(22) Filed: Apr. 7, 2026

Related U.S. Application Data
(60) Provisional application No. 63/786,644, filed on Apr. 10, 2025.

(51) Int. Cl. A61N 2/00; A61N 2/02

(52) U.S. Cl. CPC A61N 2/004; A61N 2/02

(57) ABSTRACT

A method for treatment of Atrial Fibrillation (AFib) involves using antiarrhythmic drugs in combination with pulsed electromagnetic field (PEMF) stimulation. The drugs alter conduction and refractory periods of cardiac cells and establish normal cardiac rhythm. PEMF stimulation boosts A2aAR expression in cardiac cells, enhancing the adenosine—A2aAR pathway, which reduces inflammation and restores the function of abnormal cells associated with AFib. Daily treatment for 3 to 6 months fully restores normal cardiac rhythm, eliminating the need for further medication or PEMF stimulation. A PEMF applicator includes a set of parallel straight wires placed on the patient's chest that generate a linear stimulating electric field. Additional sets of parallel wires may be used to produce multidirectional stimulation of the heart. The applicator does not have “dead” zones and provides highly uniform electric field.

### PRIORITY

This application claims the priority benefit of U.S. Provisional Application No. 63/786,644, filed on Apr. 10, 2025, which is hereby incorporated herein by reference in its entirety.

### FIELD

The invention relates to a method and apparatus for treating atrial fibrillation by activating the adenosine—A2aAR receptor pathway, known for its anti-inflammatory and tissue-restoring capabilities. Activation is achieved by applying to the heart pulsed electromagnetic field stimulation (PEMF) that increases expression of the A2aAR receptor in cardiac cells and enhances the adenosine—A2aAR anti-inflammatory signaling pathway. The enhanced pathway reduces inflammation, restores cellular architecture and functions, and facilitates the restoration of sinus rhythm of the heart.

### BACKGROUND

Atrial fibrillation (AFib) is an abnormal heart rhythm marked by rapid, irregular heartbeats in the atrial chambers. AFib reduces the efficiency of the heart's blood-pumping ability and can lead to blood clot formation, increasing the risk of stroke. Over 6 million Americans are affected by AFib today with projected prevalence of over 12 million by 2030.

In a healthy heart, the sinoatrial (SA) node, located in the right atrium and often referred to as the “natural pacemaker,” generates electrical impulses that initiate heart contractions and ensure coordinated blood flow through the heart chambers. SA node impulses generate electrical waves over the atria, causing cardiomyocytes to contract and pump blood into the ventricles.

In AFib, multiple impulses erratically fire from various atrial locations rather than the SA node, causing a chaotic rhythm. Instead of contracting properly, the atria quiver, reducing their ability to effectively pump blood into the ventricles.

The symptoms of AFib include feeling of a rapid or irregular heartbeat, palpitations both at rest and during physical activities, and chest pain that can vary from sharp and stabbing to dull ache. AFib increases the risk of stroke due to blood pooling in the left atrium, which may form clots that travel to the brain and create a stroke.

Conditions that may precipitate AFib include coronary artery disease (CAD), hypertension, rheumatic heart disease, hyperthyroidism, diabetes mellitus, inflammation, obstructive sleep apnea, and chronic obstructive pulmonary disease (COPD).

There are several pathologic changes in conductive cells of the heart that lead to AFib:

### 1. Electrical Remodeling (Ion Channel Dysfunction)

a. Action potential shortening: downregulation of L-type calcium channels leads to decreased action potential duration and a reduction in atrial refractory period, facilitating reentry circuits of AFib. Reentry circuits in atrial fibrillation (AFib) refer to abnormal electrical loops in the atria that repeatedly activate atrial tissue, causing rapid and disorganized atrial contractions.
b. Increased potassium currents: upregulation of inward rectifier potassium currents and acetylcholine-regulated K⁺ currents contribute to reentry circuits.
c. Connexin dysfunction: reduced expression and dysfunction of connexin-40 and connexin-43 in gap junctions that disrupt intercellular electrical coupling, promoting conduction heterogeneity and micro-reentry.
d. Triggered Activity: increased early afterdepolarizations (EADs) and delayed afterdepolarizations (DADs) due to intracellular calcium overload (linked to ryanodine receptor dysfunction and sarcoplasmic reticulum calcium leak).

### 2. Structural Remodeling (Fibrosis and Atrial Enlargement)

a. Fibrosis formation: excess deposition of collagen by myofibroblasts, particularly in the left atrium, disrupts normal conduction pathways and creates areas of slow conduction and reentry.
b. Atrial dilation: increased atrial size due to volume overload (e.g., from hypertension, mitral valve disease) stretches myocytes, alters gap junction function, and slows conduction.
c. Myocyte dedifferentiation and apoptosis: chronic stress and inflammation (e.g., oxidative stress, cytokine activation) lead to myocyte loss, replacement fibrosis, and disorganized conduction.

### 3. Autonomic Dysregulation

a. Enhanced sympathetic activity increases calcium influx and shortens refractoriness, promoting focal ectopic activity.
b. Vagal enhanced stimulation leads to hyperpolarization, shortening of atrial action potential duration, and increases reentry susceptibility.
c. Autonomic heterogeneity: imbalance between sympathetic and parasympathetic control leads to spatial and temporal conduction variability, favoring AFib initiation and maintenance.

### 4. Inflammation and Oxidative Stress

a. Increased reactive oxygen species (ROS): oxidative damage affects ion channels, promoting calcium leaks and afterdepolarizations.
b. Cytokine activation: elevated levels of IL-6, TNF-α, and Triggered Activity contribute to structural changes and fibrosis.
c. Endothelial dysfunction: impaired nitric oxide signaling reduces vasodilation and increases atrial ischemia risk, contributing to electrical instability.

### 5. Pulmonary Veins Triggers for AFib

a. Ectopic foci in the Pulmonary Veins: hyperactive, rapidly firing cells in the pulmonary veins can initiate AFib due to their unique electrophysiological properties (automaticity and triggered activity).
b. Anatomical and electrical remodeling of Pulmonary Vein myocytes: progressive changes make these sites a persistent source of abnormal electrical impulses.
c. The development of AFib involves a combination of electrical remodeling, structural changes (fibrosis, dilation), autonomic dysfunction, and inflammation. These alterations create a vulnerable atrial substrate that supports reentry circuits and rapid ectopic firing, ultimately sustaining AFib.

TREATMENT—Atrial fibrillation treatment depends on several factors, including the severity of symptoms, underlying causes, and risk of stroke. The main goals of treatment are rate control, rhythm control, and clot prevention.

### Classified by Severity, the AFib may be:

1. Occasional (also called paroxysmal). The symptoms come and go, usually last for a few minutes to hours, sometime for as long as a week. Symptoms might go away on their own.
2. Persistent. The irregular heartbeat is constant. The heart rhythm does not reset on its own. If symptoms occur, medical treatment is needed to correct the heart rhythm.
3. Long-standing persistent. This type of AFib is constant and lasts longer than 12 months. Medicines or a procedure are needed to correct the irregular heartbeat.
4. Permanent. In this type of atrial fibrillation, the irregular heart rhythm can't be reset. Medicines can control only the heart rate and prevent blood clots.

### Drugs Targeting Different Aspects of AFib:

1. Rate control (slowing the heart rate):
   a. Beta-blockers (e.g., metoprolol, atenolol)—commonly used, especially if the patient has high blood pressure or coronary artery disease.
   b. Calcium channel blockers (e.g., amlodipine, verapamil)—alternative to beta-blockers. Digoxin—used in some cases, especially in patients with heart failure.
2. Rhythm control: Amiodarone, Sotalol, Flecainide, Propafenone, Dofetilide
3. Clot Prevention: Anticoagulants (blood thinners):
   a. Apixaban, Rivaroxaban, Edoxaban, Dabigatran
   b. Warfarin (Coumadin): Requires regular INR monitoring, used in patients with mechanical heart valves or other specific indications.

### Procedures for Treatment of AFib:

1. Electrical Cardioversion: A controlled electric shock is delivered to the heart to restore normal rhythm. Typically used in cases of recent-onset AFib or after initial stabilization. The efficacy may vary.
2. Catheter Ablation: A minimally invasive procedure where abnormal heart tissue is destroyed to prevent AFib. Pulmonary vein isolation (PVI) is the most common ablation technique. Recommended for patients who fail medication or prefer a non-drug option.

### 3. Surgery

a. Maze Procedure: Involves creating scar tissue in the atria to block abnormal electrical signals. Done during open-heart surgery. It is today's golden standard of AFib therapy.
b. Minimally invasive surgical ablation: Alternative to catheter ablation, often done through small incisions.
c. Left Atrial Appendage Closure (LAAC):
d. Watchman Device: A catheter-based procedure that seals the left atrial appendage, reducing stroke risk in patients who cannot tolerate long-term anticoagulation.
e. Surgical implantation of a pacemaker: An implanted pacemaker is a small, battery-powered device that is surgically placed in the chest to regulate the heart rhythm.

### Limitations of AFIB Treatment

### 1. Medications:

a. Antiarrhythmic drugs: Ineffective in approximately 50% of patients. Side Effects include nausea, dizziness, fatigue. The side effects accumulate with time while the efficiency of drugs in keeping sinus rhythm fades away. Proarrhythmic risk: some drugs, like flecainide, can paradoxically worsen arrhythmias. Toxicity: Amiodarone, one of the most effective antiarrhythmic drugs, has significant toxicity risks (lung, liver, thyroid).
b. Anticoagulants (Blood Thinners): Bleeding Risk: increased risk of serious bleeding, including intracranial hemorrhage. Monitoring Needs: warfarin requires frequent INR monitoring.
   2. Catheter Ablation. Variable Long-term Success: Many patients experience AFib recurrence after ablation. Multiple procedures required: Some patients require repeat ablations. Complications: Risk of stroke, cardiac tamponade, and pulmonary vein stenosis. Cost & access issues: Expensive and not always available at all medical centers.

### 3. Devices

a. Pacemakers. Only used for AFib with bradycardia or after AV node ablation. High price, lifelong device dependency.
b. Left Atrial Appendage Occlusion Devises (e.g., Watchman): Not suitable for all AFib patients. Procedural complications like clot formation or device migration.

While current treatments for AFib have some success, new therapies are needed to improve long-term efficacy, reduce morbidity and minimize side effects.

### SUMMARY

According to experimental data, expression of A2aAR receptors can be significantly upregulated by pulsed electric fields with amplitude greater than 50 microVolt/cm. In an environment rich in extracellular adenosine, which is always the case with inflamed or stressed tissues, the upregulation of expression of A2aAR receptors leads to proportional amplification of adenosine—A2aAR signaling. In other words, in the treatment zone PEMF stimulation triggers the same anti-inflammatory response of the cells as a local increase in concentration of adenosine in the extracellular space. In either case, the amplitude of the intracellular signal from A2aAR receptors to the cellular machinery increases as well as the downstream effects of A2aAR signaling.

PEMF stimulation of A2aAR receptors provides a novel powerful therapeutic tool for treatment of various immune/inflammatory diseases. Amplified by PEMF stimulation, A2aAR signaling inhibits cytokine-producing activity in immune and parenchymal cells, suppresses chronic inflammation and restores damaged tissues. Discovered and understood only in recent decades, the adenosine A2aAR signaling pathway is one of the most powerful anti-inflammatory regulators discovered to date. Several well conducted clinical studies demonstrated that activation of A2aAR receptors leads to suppression of inflammation and promotes restoration of damaged tissues.

The adenosine—A2aAR pathway exerts strong anti-inflammatory and restorative functions. Under electric field stimulation provided by PEMF, A2aAR receptors translocate from standby positions in cytosol onto the cellular membrane where they bind with extracellular adenosine and amplify the A2aAR signaling. This phenomenon serves as a basis of a non-invasive PEMF therapy providing strong anti-inflammatory and restorative cellular actions.

An objective herein is to provide a method for non-invasive anti-inflammatory and tissue restorative treatment of Atrial Fibrillation.

Another objective is to replace the ablation of ectopic firing foci and reentry circuits in the Atria with non-invasive restoration of their normal electrical and mechanical functions by PEMF treatment.

Yet another objective is to provide a method and apparatus to deliver PEMF stimulation to the heart with a pulsed electric field of such amplitude, frequency, length, and overall duration, that facilitates the translocation of A2aAR receptors from cytosol onto cellular membranes, thereby increasing their expression on macrophages and other cardiac cells.

An additional objective is to provide a method and apparatus for uniform electric stimulation of the heart, ensuring there are no “dead zones” where the amplitude of the electric field falls below the minimum therapeutic level as is the case with a simple cylindrical coil.

The objectives are achieved in the provided example embodiments by establishing a treatment zone covering the heart with a PEMF system, which ensures a uniform distribution of the pulsed electric field across the heart. The PEMF system can offer multi-dimensional stimulation as well.

Some examples of multidirectional PEMF systems are disclosed in U.S. Pat. No. 11,571,560B2, entitled “Method and apparatus for cancer treatment” and issued to Victor Chornenky et al., which is hereby incorporated herein by reference in its entirety.

In certain disclosed examples, a method for treatment of AFib involves using antiarrhythmic drugs in combination with PEMF stimulation. The drugs alter conduction and refractory periods of cardiac cells and establish normal cardiac rhythm. PEMF stimulation boosts A2aAR expression in cardiac cells, enhancing the adenosine-A2aAR pathway, which reduces inflammation and restores the function of abnormal cells associated with AFib. Daily treatment for 3 to 6 months fully restores normal cardiac rhythm, eliminating the need for further medication or PEMF stimulation.

In certain disclosed examples, A PEMF applicator includes a set of parallel straight wires placed on the patient's chest that generate a linear stimulating electric field. Additional sets of parallel wires may be used to produce multidirectional stimulation of the heart. The applicator does not have “dead” zones and provides highly uniform electric field.

The detailed description of the therapy and preferred embodiments for the invention are described in the following paragraphs accompanying the appended drawings for people skilled in this field to well appreciate the features of the claimed invention. It is understood that the features mentioned hereinbefore and those to be commented on hereinafter may be used not only in the specified combinations, but also in other combinations or in isolation, without departing from the scope of the present invention.

### BRIEF DISCRIPTION OF THE DRAWINGS

FIG. 1 is a schematic representation of a stimulation applicator for AFib treatment of the heart according to certain example embodiments.

FIG. 2 is a schematic illustration of an applicator comprising two stimulation coils crossed at 90 degrees to each other and providing two-dimensional PEMF stimulation of the heart according to certain example embodiments.

FIG. 3 is a diagram that illustrates the time diagrams of voltage applied to the applicators (a), electric current in the coils and magnetic field in the treatment zone (b) and stimulating electric field in the treatment zone (c), according to certain example embodiments.

FIG. 4 is a diagram presenting a time diagram of electric pulses applied in directions of axes X and Y of the treatment zone according to certain example embodiments.

### DETAILED DESCRIPTION

In the following descriptions, the present invention will be explained with reference to various example embodiments; nevertheless, these example embodiments are not intended to limit the present invention to any specific example, embodiment, environment, application, or implementation described therein. Therefore, descriptions of these example embodiments are only provided for purpose illustration rather than to limit the present invention.

### Role of Macrophages in Conduction System of the Heart

Macrophages are a type of white blood cell of the innate immune system that engulf and digest pathogens, such as microbes, cellular debris and foreign substances. This process is called phagocytosis; it defends the host against infection and injury. Macrophages are found in essentially all tissues, where they patrol for potential pathogens by amoeboid movement.

In rare cases, particularly in the cardiac conduction system, a part of macrophages deviates from their normal role as sentinel cells and attach to conduction cells where they provide structural support and help maintain the cell homeostasis.

The key functions of macrophages in conduction cells include:

### 1. Electrical Conduction Support

Macrophages are actively involved in facilitating electrical conduction in the heart, particularly in the AV node.

Macrophages form gap junctions with cardiomyocytes, especially through connexin 43, enabling them to influence electrical signaling and assist in conduction where traditional pacemaker cells are less active.

### 2. Inflammation and Repair

In response to injury or stress, macrophages play a crucial role in modulating inflammation and promoting reparative processes in the conduction system. This is particularly relevant in conditions like myocarditis, atrial fibrillation, and heart block, where conduction cells are affected by inflammation.

### 3. Homeostasis and Remodeling

Macrophages help maintain the extracellular matrix and support fibroblast-cardiomyocyte interactions, which are important for structural integrity of the conduction system.

### 4. Regenerative Functions

Macrophages secrete cytokines and growth factors like IL-10, IL-4, and IGF-1, which regulate ion channel expression, support M2 anti-inflammatory macrophage conversion for tissue repair, and promote cardiomyocyte survival.

Multifactorial functions of macrophages in supporting conduction cells suggest a new approach to the treatment of AFib: stimulation of macrophages to activate the anti-inflammatory and tissue-restorative Adenosine-A2aAR pathway.

### Adenosine Anti-inflammatory and Restorative Pathway

Adenosine, a purine nucleoside generated by metabolically stressed and inflamed tissues, is recognized as a major endogenous anti-inflammatory regulator. Under normal conditions, adenosine is continuously released from cells as a product of ATP degradation. Adenosine concentration in the extracellular space is controlled by an enzyme adenosine deaminase (ADA) which breaks it down and keeps the concentration level in high-nanomolar to low-micromolar range.

Adenosine regulates the function of the innate and adaptive immune systems through targeting virtually every cell type involved in orchestrating the immune/inflammatory response. Of the four adenosine receptors (A1, A2a, A2b, A3), A2a receptors have taken center stage as the primary anti-inflammatory effectors of extracellular adenosine. The broad anti-inflammatory effect of A2a receptor activation is a result of the predominant expression of A2a receptors in all immune cells.

A2aAR activation inhibits early and late inflammatory events occurring during an immune response, which include immune cell trafficking, proliferation, proinflammatory cytokine production, and cytotoxicity. A2ARs modulation of immune cell function and inflammatory signaling shifts the balance from pro-inflammatory to anti-inflammatory responses, thereby playing a crucial role in limitation of tissue damage and facilitation of tissue restoration.

Macrophages play a central role in the immune response. At an early stage, they support inflammation by releasing cytokines that stimulate the immune system, and at the later stage, they play an important anti-inflammatory role. Macrophages that encourage inflammation are called M1 macrophages, whereas those that decrease inflammation and promote tissue restoration are called M2 macrophages. Macrophages morph into phenotypes M1 or M2 depending on the environment in which they are activated. In presence of cytokines such as IL-12 and IL-23 in initial stage of immune response, the “classically” activated macrophages are pro-inflammatory type M1, whereas at the final stage of inflammation, in presence of cytokines IL-10 produced by A2aARs actions, macrophages become “alternatively” activated into anti-inflammatory type M2 that promotes tissue healing and restoration.

Consistent with its general restorative function in tissues, A2aAR activation has been repeatedly shown to have effects that prevent excessive classical macrophage activation thereby resulting in tissue protection. In addition to its suppressive effect on the production of proinflammatory mediators, adenosine enhances the production of the anti-inflammatory cytokine IL-10, which promotes the activation of M2 macrophages involved in tissue repair.

Typically, the adenosine—A2aAR anti-inflammatory pathway is activated by elevated concentrations of extracellular adenosine. However, as was discovered lately, in joints, an electric field generated by bones and cartilage under mechanical stress (piezoelectric effect) can enhance this pathway by physical, not pharmacological means. The electric field translocates A2aAR receptors from the cytosol to cell membranes, boosting their expression and enhancing the anti-inflammatory signal that the adenosine—A2aAR pathway sends into cells.

### Pulsed Electromagnetic Field Stimulation and Adenosine

Recently it has been established by Dr. Varani et al. that the anti-inflammatory action of PEMF stimulation is attributable to its ability to increase the expression of adenosine receptors A2aAR on cellular membranes. According to their data, PEMF stimulation translocates A2aAR receptors from stand-by positions in cytosol onto the cellular membranes where they become active and available for binding with adenosine ligands.

When ligands bind to receptors, the intensity of the cellular response depends on both the concentration of ligands in the extracellular space and the concentration of receptors on the cellular membranes. As a result, the same response can be achieved by two different ways: by changing the concentration of adenosine around the cell or by changing concentration of receptors on the cellular membrane.

The essence of Dr. Varani's discovery is that PEMF stimulation can activate the adenosine-A2aAR anti-inflammatory pathway by elevating expression of A2aAR receptors without an increase of the systemic concentration of adenosine or adenosine-like drugs.

It should be mentioned that the active agent of PEMF is the electric field (EF), not the magnetic field (as stated in some publications). The magnetic component of PEMF practically does not interact with biological tissues and is used only as a carrier that deeply penetrates into tissues and a change of which generates EF (by Faraday effect). In this regard, the intensity of PEMF stimulation is measured by the amplitude of the EF pulses.

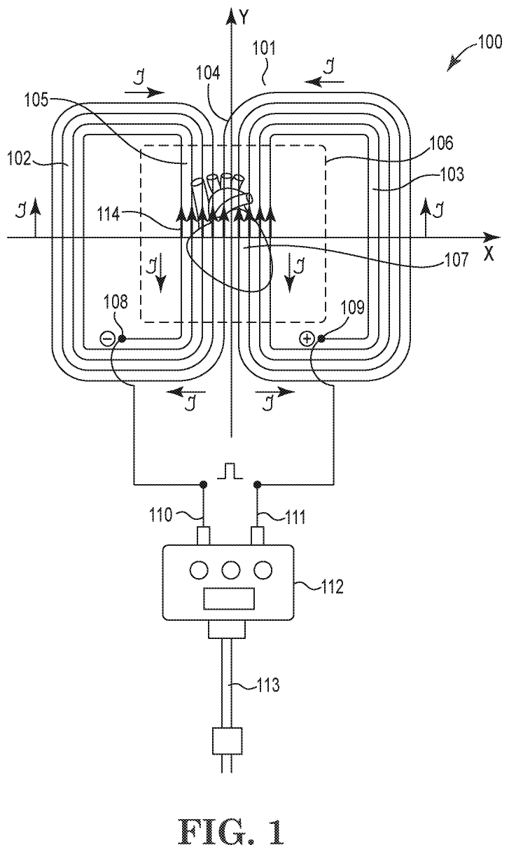

An apparatus 100 for treatment of AFib is depicted in FIG. 1. The apparatus 100 comprises electromagnetic applicator 101, which includes left coil 102 and right coil 103 connected to each other with wire 104, common for both coils, and located at the center of applicator 101. In the central portion or area of applicator 101, coils 102 and 103 form a set of parallel wires 105 that provides the electric field stimulation in the treatment zone 106, outlined by the dashed line. The target of stimulation, heart 107, is in the center of treatment zone 106.

Terminals 108 and 109 of applicator 101 are connected to the output electrodes 110 and 111 of computerized controller 112, which supplies electrical pulses to applicator 101 during operation. Power cable 113 provides the controller 112 with power and is connected to a power grid or other power source (e.g. a battery).

FIG. 1 depicts applicator 101 in which the left coil 102 and right coil 103 are connected in series with each other with a single wire 104, which is connected to electrodes 108 and 109 at its terminal ends.

In an alternative embodiment, the wire 104 can be cut into two separate wires with the left wire segment connected to electrode 109 and the right wire segment connected to electrode 108. In this alternative configuration, the left and the right coils are connected to electrodes 108 and 109 in parallel.

The controller 112 provides applicator 101 with trains of positive or negative electric pulses to create electric field stimulation in two opposite directions. FIG. 1 exemplary shows an electric pulse sent by controller 112 to terminal 108 with negative polarity and to terminal 109 with positive polarity. This pulse creates electric current in right coil 103 in counterclockwise direction and in left coil 102 clockwise direction. In the set of parallel wires 105 the electric current, as shown in FIG. 1, is directed downward along the negative direction of Y-axis, whereas electric field 114 generated by the set of wires 105, is directed upward along the positive direction of Y-axis. When a train of electric pulses sent from controller 112 to applicator 101 has reversed polarity it creates an electric field directed in the negative direction of the Y-axis (not shown in FIG. 1).

In a preferred embodiment, the apparatus for treating AFib may include not a one-dimensional applicator as described above, but two such applicators oriented at an angle of 90 degrees to each other (or a single applicator with two sets of coils arranged 90 degrees to each other). In this configuration, the two-dimensional applicator (or dual applicators) will stimulate the treatment zone with four directions with the electric field: in the plus and minus directions along the Y axis and in the plus and minus directions along the X axis.

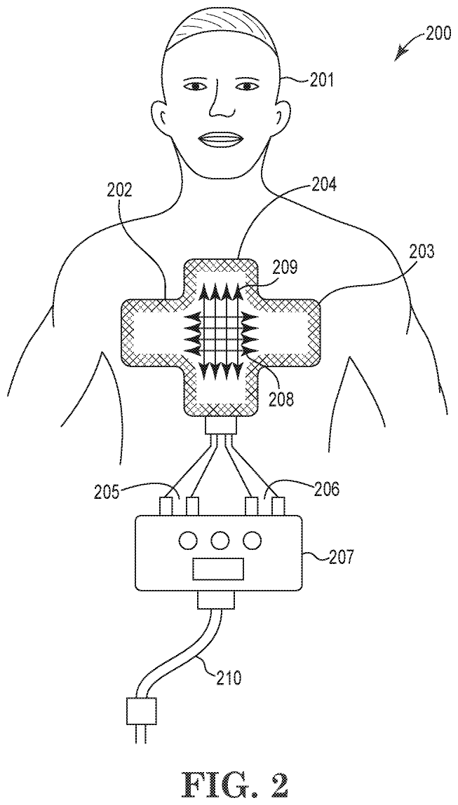

FIG. 2. schematically shows a two-dimensional applicator apparatus 200 for treatment of AFib. Numeral 201 is designated for the patient. The schematically depicted two-dimensional applicator 202, having a flexible fabric cover for coils, is placed on the patient's chest close to the heart. The flexible fabric cover of applicator 202 allows it to conform to the patient's body, ensuring close contact and effective delivery of the electric field stimulation.

The two-dimensional applicator 202 comprises a first one-dimensional applicator 203 (or coil set), positioned horizontally and a second one-dimensional applicator 204 (or coil set), positioned vertically. The terminals of the horizontally positioned applicator 203 portion are connected to output electrodes 205 of controller 207, designed to generate trains of electric pulses of different polarity to provide two directions of electric field stimulation along the X-axis. The terminals of vertically positioned actuator 204 portion are connected to output electrodes 206 of controller 207 that separately generate trains of electric pulses of different polarity to provide two directions of electric field stimulation along the Y-axis. The electric field stimulation provided along the X-axis is shown by numeral 208, electric field stimulation provided along the Y-axis is shown by numeral 209. Numeral 210 is designated as a power cable which can be connected to a power grid, battery or other power source.

Additionally, the apparatus 100, 200 disclosed herein is designed to be user-friendly and safe. The power supply is equipped with safety mechanisms to prevent any electrical hazards, and the controller 112, 207 has built-in features to monitor the operation of the device to ensure that the parameters of the electric pulses are within the therapeutic range.

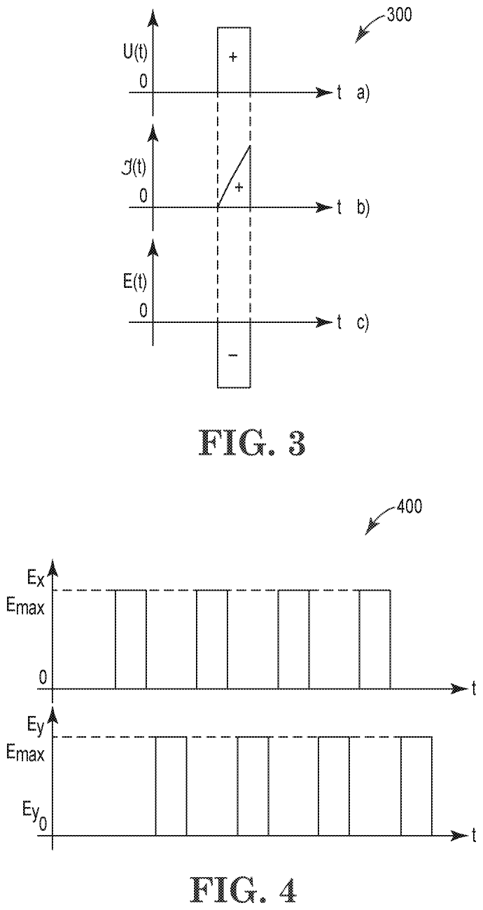

FIG. 3 shows a diagram 300 of a voltage applied to an applicator (a), electrical current and magnetic field in the applicator (b) and the electric field in the treatment zone (c) during operation. The voltage pulse (a) generated by controller 112, 207 on electrodes 105, 205 or electrodes 106, 206 is preferentially rectangular (DC pulse). The graph of the current in an applicator (b) is an exponential curve close to a straight-line. Graph (c) represents a therapeutic electric pulse in the treatment zone.

FIG. 4 is a diagram 400 that illustrates the pulses of electric fields Eₓ and E_{y} in the treatment zone, generated by horizontal and vertical applicators 204 and 203 (or coil pairs). The pulses alternate at the same frequency without overlapping, which allows for a shorter operation time with equal field exposure. During operation, pulse polarity changes midway to ensure equal stimulation in all four directions: +Eₓ, −Ex, +E_{y}, and −E_{y}.

The particular mode of treatment of AFib with PEMF stimulation depends on the stage of AFib which are: Paroxysmal, Persistent, Long-standing Persistent and Permanent.

According to disclosed invention, paroxysmal AFib is treated during the time periods when a patient is free from AFib symptoms. For treatment, the applicator, such as described herein, is placed on the center of patient's chest to create a pulsed electric field stimulation in the heart.

The treatment regimen comprises 5 to 10 minutes of stimulation in each direction of the electric field, resulting in a total of 20 to 40 minutes per session. 2-3 sessions should be performed daily with 3 hours between sessions for resetting cellular condition of A 2aAR receptors. The total recommended duration of the treatment is 3 to 6 months.

Persistent and long-standing persistent AFib are treated with medications that control the patient's heart rate and rhythm, such as calcium channel blockers and beta blockers. Over time, ectopic foci initiating AFib and re-entering pathways tend to become more stable and resilient, making their treatment challenging, following the saying “AFib begets AFib”. Restoration of their normal function is more effective during periods of calm, when they do not actively participate in AFib electric activity and engage solely in the restorative processes activated by the Adenosine-—2aARs pathway.

In another words, the efficient treatment of persistent and long-standing persistent AFib is provided by a combination of antiarrhythmic drugs and PEMF stimulation administered for several months.

Electrical cardioversion is another way of restoring normal heart rhythm that can be followed by PEMF treatment. The recommended regiment of treatment by PEMF stimulation in both cases is the same as for paroxysmal AFib as described above.

In the case of permanent AFib, PEMF stimulation still can be beneficial to the patients. It inhibits inflammation and stops fibrosis, which is the main driving force of the detrimental remodeling of the heart and the main cause of evolution of AFib to Heart Failure.

Another state of atrial fibrillation is postoperative atrial fibrillation (POAF), the most common arrhythmia after coronary artery bypass grafting (CABG). It is usually treated with antiarrhythmic drugs. The use of these drugs in combination with PEMF provides two additional benefits to the patient: it reverses the structural and electrical changes in the cells associated with AFib, thereby reducing the need for long-term drug therapy, and accelerates wound healing after surgery.

### Atrial Fibrosis

Atrial fibrosis, primarily caused by cardiac fibroblast activation and differentiation into myofibroblasts, leads to worsening of AFib symptoms. These myofibroblasts secrete excess ECM proteins like collagen type I and III. Among various protective actions, the adenosine A2aAR pathway has been recognized for its anti-fibrotic properties, particularly in the cardiovascular system and AFib-induced fibrosis. A2aAR activation plays a protective role by modulating fibroblast activity, reducing collagen deposition, and inhibiting pro-fibrotic signaling pathways. A2aAR activation reduces fibroblast proliferation by increasing cyclic adenosine monophosphate (cAMP) levels, leading to inhibition of TGF-β1 signaling, a major pro-fibrotic pathway, decrease of production of fibrotic proteins, such as collagen I, collagen III, and fibronectin, excessive extracellular matrix deposition and atrial stiffening promoting sustained AFib reentry circuits. The activation of A2aAR pathway in cardiomyocytes and conductive cells leads to inhibition of fibrosis and restoration of their architecture and functions.

### A Case Study

Patient: 82- year-old male with post-Covid- 19 AFib, diabetes (HbA 1 6.4%). Pre-ablation medications: Amlodipine for blood pressure and heart rate, Warfarin for thrombosis prevention, Berberine for blood sugar control. Heart rate: 50 bpm.

The catheter ablation, done 5 months after the start of atrial fibrillation, failed. Then Sotalol was prescribed which successfully controlled the heart rhythm. Six months later, PEMF treatment started along with Amlodipine and Sotalol. After 4 months, Amlodipine and Sotalol were paused for three days and ECG scan demonstrated sinus rhythm. Amlodipine and Sotalol were then discontinued indefinitely.

During three months after discontinuity of Amlodipine and Sotalol, PEMF treatment was continued, the sinus rhythm stayed stable at rest and during physical activity, the heart rate at rest was 65-70 bpm, during moderate exercise 80-105 bpm. No signs of AFib for 10 months!

While the invention has been described in connection with what is presently considered to be the most practical and preferred example embodiments, it will be apparent to those of ordinary skill in the art that the invention is not to be limited to the disclosed example embodiments. It will be readily apparent to those of ordinary skill in the art that many modifications and equivalent arrangements can be made thereof without departing from the spirit and scope of the present disclosure, such scope to be accorded the broadest interpretation of the appended claims so as to encompass all equivalent structures and products.

It is also within the scope of the invention to combine features, functions, advantages and aspects of the various embodiments described herein. Thus, the embodiments of the invention may comprise combinations of aspects of any one or more of these exemplary embodiments.

For purposes of interpreting the claims for the present invention, it is expressly intended that the provisions of Section 112, sixth paragraph of 35 U.S.C. are not to be invoked unless the specific terms “means for” or “step for” are recited in a claim.

What is claimed is:

1. A method of treating atrial fibrillation in a patient, the method comprising:
applying a pulsed electromagnetic field (PEMF) stimulation to a treatment volume within the patient containing a heart of the patient sufficient to increase expression of A2aAR receptor in cardiac cells to enhance an adenosine—A2aAR anti-inflammatory signaling pathway such that inflammation in the heart is reduced.

2. The method of claim 1 wherein the PEMF stimulation is periodically applied to the treatment volume until sinus rhythm of the heart is restored.

3. The method of claim 1, wherein the PEMF stimulation is applied to the treatment volume in multiple different spatial directions.

4. The method of claim 1, wherein the PEMF stimulation is applied to the treatment volume in a first spatial direction and in a second spatial direction, wherein the second spatial direction is perpendicular to the first spatial direction.

5. The method of claim 1, wherein the treatment volume is free from electrical field dead zones.

6. The method of claim 1, wherein the application of the PEMF stimulation is provided via an applicator applied to a chest of the patient, the applicator including a first coil and a second coil coupled to a controller to create a counterclockwise current in the first coil and a clockwise current in the second coil, wherein the first coil and second coil are arranged to create an electrical field in the treatment volume along a first axial orientation.

7. The method of claim 6, further comprising the controller applying a series of square wave electrical pulses to the first and second coils, wherein the series of square wave electrical pulses are alternatingly positive and negative polarities.

8. The method of claim 6, wherein the applicator further includes a third coil and a fourth coil coupled to the controller to create a counterclockwise current in the third coil and a clockwise current in the fourth coil, wherein the third coil and fourth coil are arranged to create an electrical field in the treatment volume along a second axial orientation that is perpendicular to the first axial direction.

9. The method of claim 1, wherein the PEMF stimulation is applied to the patient in multiple sessions per day of 20-40 minutes each session.

10. The method of claim 9, wherein each of the multiple sessions per day are spaced apart by a plurality of hours.

11. The method of claim 10, wherein the multiple sessions per day are continued for a period of three to six months.

12. A method of activating adenosine A2aAR receptors in cardiac cells of a heart of a patient experiencing atrial fibrillation, the method comprising:
applying a pulsed electromagnetic field (PEMF) stimulation to the heart of the patient sufficient to increase expression of A2aAR adenosine receptors in the cardiac cells of the patient such that inflammation in the cardiac cells is reduced.

13. The method of claim 12, wherein the PEMF stimulation is applied to the heart in multiple different spatial directions.

14. The method of claim 12, wherein the application of the PEMF stimulation is provided via an applicator applied to a chest of the patient, the applicator including a first coil and a second coil coupled to a controller to create a counterclockwise current in the first coil and a clockwise current in the second coil, wherein the first coil and second coil are arranged to create an electrical field in the heart along a first axial orientation.

15. The method of claim 14, further comprising the controller applying a series of electrical pulses to the first and second coils with alternatingly positive and negative polarities.

16. The method of claim 14, wherein the applicator further includes a third coil and a fourth coil coupled to the controller to create a counterclockwise current in the third coil and a clockwise current in the fourth coil, wherein the third coil and fourth coil are arranged to create an electrical field in the heart along a second axial orientation that is perpendicular to the first axial direction.

17. A pulsed electric field stimulation apparatus for treatment of atrial fibrillation of a heart of a patient, the heart located within a treatment volume, the apparatus comprising:
an applicator;
a first coil disposed in the applicator and including a first straight wire segment;
a second coil disposed in the applicator and including a second straight wire segment;
a controller electrically coupled to first coil to create a counterclockwise current in the
first coil and electrically coupled to the second coil to create a clockwise current in the second coil,
wherein the first coil and second coil are arranged such that the first straight wire segment is oriented parallel to the second straight wire segment to create a linear stimulation electrical field along a first axis in the treatment volume.

18. The apparatus of claim 17, wherein the applicator is configured for application to a chest of patient.

19. The apparatus of claim 17, further comprising:
a third coil disposed in the applicator and including a third straight wire segment;
a fourth coil disposed in the applicator and including a fourth straight wire segment;
wherein the controller is electrically coupled to third coil to create a counterclockwise current in the third coil and electrically coupled to the fourth coil to create a clockwise current in the fourth coil,
wherein the third coil and fourth coil are arranged such that the third straight wire segment is oriented parallel to the fourth straight wire segment to create a linear stimulation electrical field along a second axis in the treatment volume, and
wherein the second axis is oriented perpendicular to the first axis.

20. The apparatus of claim 17, wherein the controller is configured to apply a series of electrical pulses to each of the first and second coils with alternatingly positive and negative polarities.
